(19) United States
(12) Patent Application Publication
(10) Pub. No.: US 2026/0306495 A1
(43) Pub. Date: Oct. 8, 2026

(54) COMBINATION OF CAPIVASERTIB, A CDK4/6 INHIBITOR AND FULVESTRANT FOR USE IN THE TREATMENT OF BREAST CANCER

(71) Applicant: AstraZeneca AB, Södertälje (SE)

(72) Inventors: Simon BARRY, Cambridge (GB); Elza DE BRUIN, Cambridge (GB); Andrew FOXLEY, Cambridge (GB); Amy MCDONOUGH, Cambridge (GB); Gaia SCHIAVON, Cambridge (GB)

(21) Appl. No.: 19/481,328

(22) PCT Filed: May 3, 2024
(86) PCT No.: PCT/EP2024/062253

§ 371 Date: Nov. 4, 2025

Related U.S. Application Data
(60) Provisional application No. 63/500,348, filed on May 5, 2023.
(60) Provisional application No. 63/568,170, filed on Mar. 21, 2024.

(51) Int. Cl. A61K 31/519; A61K 31/567; A61P 35/00

(52) U.S. Cl. CPC A61K 31/519; A61K 31/567; A61P 35/00

(57) ABSTRACT

The specification relates to therapeutic combinations of capivasertib, a CDK4/6 inhibitor (such as palbociclib, ribociclib or abemaciclib) and fulvestrant that are useful for treating breast cancer, and to methods of treating breast cancer patients with combinations of capivasertib, a CDK4/6 inhibitor (such as palbociclib, ribociclib or abemaciclib) and fulvestrant.

### CROSS-REFERENCE TO RELATED APPLICATIONS

This application claims priority to U.S. Provisional Patent Application No. 63/500,348, filed 5 May 2023; and U.S. Provisional Patent Application No. 63/568,170, filed 21 Mar. 2024; the disclosures of which are incorporated by reference herein.

### FIELD

The present disclosure relates to therapeutic combinations of capivasertib, a CDK4/6 inhibitor and fulvestrant that are useful for treating breast cancer, and to methods of treating breast cancer patients with combinations of capivasertib, a CDK4/6 inhibitor and fulvestrant.

### BACKGROUND

More than two-thirds of patients with advanced breast cancer (ABC) have hormone receptor-positive/human epidermal growth factor receptor 2-negative (HR+/HER2−) tumours (Howlader N et al., J Natl Cancer Inst 2014; 106). Endocrine therapy (ET) is the core treatment for patients with HR+/HER2− ABC, most commonly an aromatase inhibitor (AI), tamoxifen (a selective estrogen receptor modulator (SERM)), or fulvestrant (a selective estrogen receptor degrader (SERD)). Many patients additionally receive a cyclin-dependent kinase (CDK) 4/6 inhibitor in combination with ET, which prolongs life compared with ET alone (Spring L M et al. Lancet 2020; 395:817-27; Cardoso F et al. Ann Oncol 2020; 31:1623-49). However, patients eventually develop resistance to ET plus CDK4/6 inhibitor combination therapy, which results in disease progression (Spring L M et al. Lancet 2020; 395:817-27).

Overcoming therapy resistance is a major challenge, partially because it is associated with several different mechanisms. The phosphoinositide 3-kinase/protein kinase/phosphatase and tensin homologue (PI3K/AKT/PTEN) pathway regulates cell growth, proliferation and survival (Brown J S, Banerji U. Pharmacol Ther 2017; 172:101-15). Aberrant activation of the PI3K/AKT/PTEN pathway is a key compensatory mechanism driving multi-drug resistance in cancers in patients receiving ET with a CDK4/6 inhibitor (Spring L M et al. Lancet 2020; 395:817-27; O'Leary B et al. Cancer Discov 2018; 8:1390-403; Costa C et al. Cancer Discov 2020; 10:72-85; Wander S A et al. Cancer Discov 2020; 10:1174-93).

In view of the unmet need for overcoming resistance to ET plus CDK4/6 inhibitor combination therapy, the CAPItello-292 Phase Ib/III study (ClinicalTrials.gov Identifier: NCT04862663) aims to evaluate the efficacy, safety and the degree of added benefit of capivasertib (a potent, selective pan-AKT kinase inhibitor) combined with CDK4/6i and fulvestrant in participants with locally advanced (inoperable) or metastatic HR+/HER2− breast cancer. The CAPItello-292 study aims to establish a recommended Phase III doses (RP3D) and schedule for the triplet combinations (capivasertib+CDK4/6i (palbociclib, ribociclib or abemaciclib)+fulvestrant). Results from the CAPItello-292 clinical study, and therapeutic dosage regimens for the triplet combinations (capivasertib+CDK4/6i (palbociclib, ribociclib or abemaciclib)+fulvestrant) are described herein.

### SUMMARY

A first aspect of the disclosure provides a method of treating a patient having hormone receptor-positive (HR+), HER2-negative locally advanced or metastatic breast cancer, comprising administering to the patient a combination therapy comprising a first amount of capivasertib, a second amount of a CDK4/6 inhibitor and a third amount of fulvestrant, wherein the first amount, second amount and third amount together comprise a therapeutically effective amount.

A second aspect of the disclosure provides a combination therapy for use in treating a patient having hormone receptor-positive (HR+), HER2-negative locally advanced or metastatic breast cancer, wherein the combination therapy comprises capivasertib, a CDK4/6 inhibitor and fulvestrant.

A third aspect of the disclosure provides a use of capivasertib in the manufacture of a medicament for treating a patient having hormone receptor-positive (HR+), HER2-negative locally advanced or metastatic breast cancer with a combination therapy comprising capivasertib, a CDK4/6 inhibitor and fulvestrant.

A fourth aspect of the disclosure provides a kit comprising (a) capivasertib, and (b) instructions for the use of capivasertib to treat a patient having hormone receptor-positive (HR+), HER2-negative locally advanced or metastatic breast cancer with a combination therapy comprising capivasertib, a CDK4/6 inhibitor and fulvestrant.

A fifth aspect of the disclosure provides a kit comprising (a) a combination therapy comprising capivasertib, a CDK4/6 inhibitor and fulvestrant, and (b) instructions for the use of the combination therapy in the treatment of a patient having hormone receptor-positive (HR+), HER2-negative locally advanced or metastatic breast cancer.

Further aspects of the disclosure will be apparent to one skilled in the art from reading this specification.

### DESCRIPTION OF THE DRAWINGS

FIG. 1 shows circulating tumour DNA (ctDNA) response on day 1, cycle 2 in patients in the CAPItello-292 study treated with the following combination therapy administered over a 28-day cycle: 400 mg of capivasertib administered orally twice daily on days 1 to 4, 8 to 11, 15 to 18, and 22 to 25; 125 mg of palbociclib administered orally once daily on days 1 to 21; and 500 mg of fulvestrant is administered intramuscularly once daily on days 1 and 15 of the first 28-day cycle, and once on day 1 of the 28-day cycle thereafter. ctDNA was evaluated in participants with available samples (n=10).

FIG. 2 shows preliminary response data in patients in the CAPItello-292 study treated with the following combination therapy administered over a 28-day cycle: 400 mg of capivasertib administered orally twice daily on days 1 to 4, 8 to 11, 15 to 18, and 22 to 25; 125 mg of palbociclib administered orally once daily on days 1 to 21; and 500 mg of fulvestrant is administered intramuscularly once daily on days 1 and 15 of the first 28-day cycle, and once on day 1 of the 28-day cycle thereafter. Evidence of clinical activity in this heavily pre-treated population at this dosing schedule is shown. Confirmed partial responses to the triplet combination were observed, including in patients previously treated with CDK4/6i and/or fulvestrant.

### DETAILED DESCRIPTION

### Definitions

As used herein, the term “patient” refers to a mammal, optionally a human.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the patient and disease condition being treated (e.g., the weight, age and gender of the patient), the severity of the disease condition, the manner of administration, etc. The term also applies to a dose that will induce a particular response in target cells. The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “treat,” “treating,” and “treatment” refer to at least partially alleviating, inhibiting, preventing and/or ameliorating a condition, disorder, or disease, such as advanced breast cancer. The effectiveness of treatment of advanced breast cancer can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging progression free survival (PFS).

The term “combination therapy” can refer to simultaneous, separate, or sequential administration of two or more therapeutic agents. In one embodiment, “combination” can refer to simultaneous administration (e.g., administration of multiple agents in a single dosage form). In another embodiment, “combination” refers to separate administration (e.g., administration of multiple agents in separate dosage forms, but at substantially the same time). In a further embodiment, “combination” refers to sequential administration (e.g., where a first therapeutic agent is administered, followed by a delay, followed by administration of a second or further therapeutic agent). Where the administration is sequential or separate, the delay in administering the later component should be neither too long nor too short, so as not to lose the benefit of the combination.

The terms “co-administration,” “in combination with,” “simultaneous,” and “concurrent,” as used herein, encompass administration of two or more therapeutic agents to a patient and include simultaneous administration in separate compositions, administration at different times in separate compositions, or administration in a composition in which two or more therapeutic agents are present.

The terms “first amount”, “second amount” and “third amount” are used to refer to the amount of each component in the combinations described herein and do not necessarily reflect order of dosing.

The term “RECIST v1.1” refers to the Response Evaluation Criteria in Solid Tumours (RECIST) version 1.1 criteria described in Eisenhauer et al., European Journal of Cancer (2009) 45:228-247.

### Methods

In one embodiment, there is provided a method of treating a patient having hormone receptor-positive (HR+), HER2-negative locally advanced or metastatic breast cancer, comprising administering to the patient a combination therapy comprising a first amount of capivasertib, a second amount of a CDK4/6 inhibitor and a third amount of fulvestrant, wherein the first amount, second amount and third amount together comprise a therapeutically effective amount.

In one embodiment, there is provided a combination therapy for use in treating a patient having hormone receptor-positive (HR+), HER2-negative locally advanced or metastatic breast cancer, wherein the combination therapy comprises capivasertib, a CDK4/6 inhibitor and fulvestrant.

In one embodiment, there is provided a use of capivasertib in the manufacture of a medicament for treating a patient having hormone receptor-positive (HR+), HER2-negative locally advanced or metastatic breast cancer with a combination therapy comprising capivasertib, a CDK4/6 inhibitor and fulvestrant.

In one embodiment, there is provided a kit comprising (a) capivasertib, and (b) instructions for the use of capivasertib to treat a patient having hormone receptor-positive (HR+), HER2-negative locally advanced or metastatic breast cancer with a combination therapy comprising capivasertib, a CDK4/6 inhibitor and fulvestrant.

In one embodiment, there is provided a kit comprising (a) a combination therapy comprising capivasertib, a CDK4/6 inhibitor and fulvestrant, and (b) instructions for the use of the combination therapy in the treatment of a patient having hormone receptor-positive (HR+), HER2-negative locally advanced or metastatic breast cancer.

### Breast Cancer

In one embodiment, the breast cancer is histologically confirmed HR+/HER2− breast cancer determined from the most recent tumour sample (primary or metastatic) per the American Society of Clinical Oncology and College of American Pathologists guideline (Hammond M E H et al., J Clin Oncol 2010, 28 (16), 2784-95; Erratum in: J Clin Oncol. 2010, 28 (21), 3543; Wolff A C et al., Arch Pathol Lab Med 2018, 142 (11), 1364-82). To fulfil the requirement of HR+ disease, a breast cancer must express estrogen receptor (ER) with or without co-expression of progesterone receptor. Therefore, tumours must be:
(a) Estrogen receptor positive (ER+) defined as ≥1% of tumour cells stain positive for ER on immunohistochemistry (IHC) or, if no percentage is available, then an Allred IHC score of ≥⅜,
(b) Progesterone receptor positive defined as ≥1% of tumour cells stain positive for progesterone receptor on IHC or, if no percentage is available, then an Allred IHC score of ≥⅜; or progesterone receptor negative defined as <1% of tumour cells stain positive for progesterone receptor on IHC or, if no percentage is available, then an Allred IHC score of ≤ 2/8; or progesterone receptor unknown, and
(c) HER2-defined as 0 or 1+ intensity on IHC, or 2+ intensity on IHC and no evidence of amplification on in situ hybridisation (ISH).

In one embodiment, the breast cancer is HR+, HER2-negative locally advanced (inoperable) or metastatic breast cancer. In one embodiment, the breast cancer is HR+, HER2-negative locally advanced (inoperable) breast cancer. In one embodiment, the breast cancer is HR+, HER2-negative metastatic breast cancer.

### Combination Therapy

Capivasertib (also known as AZD5363 and by the chemical name of(S)-4-amino-N-(1-(4-chlorophenyl)-3-hydroxypropyl)-1-(7H-pyrrolo[2,3-d]pyrimidin-4-yl) piperidine-4-carboxamide) is a potent, selective adenosine triphosphate (ATP)-competitive inhibitor of all three AKT isoforms (AKT1/2/3). It is an investigational oral treatment currently in Phase III trials for the treatment of multiple subtypes of breast cancer and prostate cancer.

In one embodiment, the first amount of capivasertib administered to the patient is 320 mg twice daily. In one embodiment, the first amount of capivasertib administered to the patient is 400 mg twice daily.

In one embodiment, 320 mg of capivasertib is orally administered to the patient twice daily; 4 days on, 3 days off. In one embodiment, 400 mg of capivasertib is orally administered to the patient twice daily; 4 days on, 3 days off.

In one embodiment, capivasertib is administered to the patient over a 28-day cycle in which 320 mg of capivasertib is orally administered to the patient twice daily on days 1 to 4, 8 to 11, 15 to 18, and 22 to 25. In one embodiment, capivasertib is administered to the patient over a 28-day cycle in which 400 mg of capivasertib is orally administered to the patient twice daily on days 1 to 4, 8 to 11, 15 to 18, and 22 to 25.

Cyclin-dependent kinase 4 and 6 (CDK4/6) inhibitors are known in the art and include, for example, palbociclib (known by the chemical name 6-acetyl-8-cyclopentyl-5-methyl-2-{[5-(1-piperazinyl)-2-pyridinyl]amino}pyrido[2,3-d]pyrimidin-7 (8H)-one, and sold under the brand name IBRANCE among others), ribociclib (known by the chemical name 7-cyclopentyl-N,N-dimethyl-2-{[5-(1-piperazinyl)-2-pyridinyl]amino}-7H-pyrrolo[2,3-d]pyrimidine-6-carboxamide, and sold under the brand names KISQALI and KRYXANA) and abemaciclib (known by the chemical name N-{5-[(4-ethyl-1-piperazinyl)methyl]-2-pyridinyl}-5-fluoro-4-(4-fluoro-1-isopropyl-2-methyl-1H-benzimidazol-6-yl)-2-pyrimidinamine, and sold under the brand name VERZENIO among others)). In embodiments, the CDK4/6 inhibitor is any molecule which binds to and inhibits the activity of CDK4 and CDK6 (for example having a pIC50 of >4.5, >5, >6, >7, >8 or >9 vs. CDK4 and CDK6 when tested in a standard potency assay, for example as described in WO03062236, WO2010020675 or WO2010075074). In one embodiment, the CDK4/6 inhibitor is selected from palbociclib, ribociclib, and abemaciclib.

In one embodiment, the CDK4/6 inhibitor is palbociclib.

In one embodiment, 75 mg of palbociclib is orally administered to the patient once daily. In one embodiment, 100 mg of palbociclib is orally administered to the patient once daily. In one embodiment, 125 mg of palbociclib is orally administered to the patient once daily.

In one embodiment, palbociclib is administered to the patient over a 28-day cycle in which 75 mg of palbociclib is orally administered to the patient once daily on days 1 to 21. In one embodiment, palbociclib is administered to the patient over a 28-day cycle in which 100 mg of palbociclib is orally administered to the patient once daily on days 1 to 21. In one embodiment, palbociclib is administered to the patient over a 28-day cycle in which 125 mg of palbociclib is orally administered to the patient once daily on days 1 to 21. In one embodiment, palbociclib is administered to the patient with food.

In one embodiment, 125 mg of palbociclib is orally administered to the patient once daily, and the patient is not a Japanese patient. In one embodiment, palbociclib is administered to the patient over a 28-day cycle in which 125 mg of palbociclib is orally administered to the patient once daily on days 1 to 21, and the patient is not a Japanese patient.

In one embodiment, 100 mg of palbociclib is orally administered to the patient once daily, and the patient is a Japanese patient. In one embodiment, palbociclib is administered to the patient over a 28-day cycle in which 100 mg of palbociclib is orally administered to the patient once daily on days 1 to 21, and the patient is a Japanese patient.

In one embodiment, 125 mg of palbociclib is orally administered to the patient once daily, and the patient is not an Asian patient. In one embodiment, palbociclib is administered to the patient over a 28-day cycle in which 125 mg of palbociclib is orally administered to the patient once daily on days 1 to 21, and the patient is not an Asian patient.

In one embodiment, 100 mg of palbociclib is orally administered to the patient once daily, and the patient is an Asian patient. In one embodiment, palbociclib is administered to the patient over a 28-day cycle in which 100 mg of palbociclib is orally administered to the patient once daily on days 1 to 21, and the patient is an Asian patient.

In one embodiment, the CDK4/6 inhibitor is ribociclib.

In one embodiment, 200 mg of ribociclib is orally administered to the patient once daily. In one embodiment, 400 mg of ribociclib is orally administered to the patient once daily. In one embodiment, 600 mg of ribociclib is orally administered to the patient once daily.

In one embodiment, ribociclib is administered to the patient over a 28-day cycle in which 200 mg of ribociclib is orally administered to the patient once daily on days 1 to 21. In one embodiment, ribociclib is administered to the patient over a 28-day cycle in which 400 mg of ribociclib is orally administered to the patient once daily on days 1 to 21. In one embodiment, ribociclib is administered to the patient over a 28-day cycle in which 600 mg of ribociclib is orally administered to the patient once daily on days 1 to 21.

In one embodiment, the CDK4/6 inhibitor is abemaciclib.

In one embodiment, 50 mg of abemaciclib is orally administered to the patient twice daily. In one embodiment, 100 mg of abemaciclib is orally administered to the patient twice daily. In one embodiment, 150 mg of abemaciclib is orally administered to the patient twice daily.

In one embodiment, abemaciclib is administered to the patient over a 28-day cycle in which 50 mg of abemaciclib is orally administered to the patient twice daily on days 1 to 28. In one embodiment, abemaciclib is administered to the patient over a 28-day cycle in which 100 mg of abemaciclib is orally administered to the patient twice daily on days 1 to 28. In one embodiment, abemaciclib is administered to the patient over a 28-day cycle in which 150 mg of abemaciclib is orally administered to the patient twice daily on days 1 to 28.

Fulvestrant, sold under the brand name FASLODEX among others, and known by the chemical name 7α-[9-[(4,4,5,5,5-Pentafluoropentyl)-sulfinyl]nonyl]estra-1,3,5(10)-triene-3,17β-diol, is a selective estrogen receptor degrader (SERD) used to treat ER+ metastatic breast cancer (including ER+/HER2-negative breast cancers). In one embodiment, fulvestrant is administered to the patient by intramuscular injection and is provided in a pre-filled syringe containing 250 mg fulvestrant in 5 ml solution. In one embodiment, 500 mg fulvestrant is intramuscularly administered to the patient once monthly. In one embodiment, 500 mg fulvestrant is intramuscularly administered to the patient once every 28 days. In one embodiment, an additional 500 mg dose of fulvestrant is intramuscularly administered to the patient two weeks after the initial dose.

In one embodiment, fulvestrant is administered to the patient over a 28-day cycle in which 500 mg of fulvestrant is intramuscularly administered to the patient once daily on days 1 and 15 of the first 28-day cycle, and once on day 1 of the 28-day cycle thereafter.

In one embodiment, 400 mg of capivasertib is orally administered to the patient twice daily; 4 days on, 3 days off; 125 mg of palbociclib is orally administered to the patient once daily for 21 consecutive days followed by 7 days off; 500 mg of fulvestrant is intramuscularly administered to the patient once every 28 days, and optionally an additional 500 mg dose of fulvestrant is intramuscularly administered to the patient two weeks after the initial dose.

In one embodiment, 400 mg of capivasertib is orally administered to the patient twice daily; 4 days on, 3 days off; 100 mg of palbociclib is orally administered to the patient once daily for 21 consecutive days followed by 7 days off; 500 mg of fulvestrant is intramuscularly administered to the patient once every 28 days, and optionally an additional 500 mg dose of fulvestrant is intramuscularly administered to the patient two weeks after the initial dose.

In one embodiment, 400 mg of capivasertib is orally administered to the patient twice daily; 4 days on, 3 days off; 125 mg of palbociclib is orally administered to the patient once daily for 21 consecutive days followed by 7 days off; 500 mg of fulvestrant is intramuscularly administered to the patient once every 28 days, and optionally an additional 500 mg dose of fulvestrant is intramuscularly administered to the patient two weeks after the initial dose, and the patient is not a Japanese patient.

In one embodiment, 400 mg of capivasertib is orally administered to the patient twice daily; 4 days on, 3 days off; 100 mg of palbociclib is orally administered to the patient once daily for 21 consecutive days followed by 7 days off; 500 mg of fulvestrant is intramuscularly administered to the patient once every 28 days, and optionally an additional 500 mg dose of fulvestrant is intramuscularly administered to the patient two weeks after the initial dose, and the patient is a Japanese patient.

In one embodiment, 400 mg of capivasertib is orally administered to the patient twice daily; 4 days on, 3 days off; 125 mg of palbociclib is orally administered to the patient once daily for 21 consecutive days followed by 7 days off; 500 mg of fulvestrant is intramuscularly administered to the patient once every 28 days, and optionally an additional 500 mg dose of fulvestrant is intramuscularly administered to the patient two weeks after the initial dose, and the patient is not an Asian patient.

In one embodiment, 400 mg of capivasertib is orally administered to the patient twice daily; 4 days on, 3 days off; 100 mg of palbociclib is orally administered to the patient once daily for 21 consecutive days followed by 7 days off; 500 mg of fulvestrant is intramuscularly administered to the patient once every 28 days, and optionally an additional 500 mg dose of fulvestrant is intramuscularly administered to the patient two weeks after the initial dose, and the patient is an Asian patient.

In one embodiment, the combination therapy is administered to the patient over a 28-day cycle in which 400 mg of capivasertib is orally administered to the patient twice daily on days 1 to 4, 8 to 11, 15 to 18, and 22 to 25; 125 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

In one embodiment, the combination therapy is administered to the patient over a 28-day cycle in which 400 mg of capivasertib is orally administered to the patient twice daily on days 1 to 4, 8 to 11, 15 to 18, and 22 to 25; 100 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

In one embodiment, the combination therapy is administered to the patient over a 28-day cycle in which 400 mg of capivasertib is orally administered to the patient twice daily on days 1 to 4, 8 to 11, 15 to 18, and 22 to 25; 125 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter, and the patient is not a Japanese patient.

In one embodiment, the combination therapy is administered to the patient over a 28-day cycle in which 400 mg of capivasertib is orally administered to the patient twice daily on days 1 to 4, 8 to 11, 15 to 18, and 22 to 25; 100 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter, and the patient is a Japanese patient.

In one embodiment, the combination therapy is administered to the patient over a 28-day cycle in which 400 mg of capivasertib is orally administered to the patient twice daily on days 1 to 4, 8 to 11, 15 to 18, and 22 to 25; 125 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter, and the patient is not an Asian patient.

In one embodiment, the combination therapy is administered to the patient over a 28-day cycle in which 400 mg of capivasertib is orally administered to the patient twice daily on days 1 to 4, 8 to 11, 15 to 18, and 22 to 25; 100 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter, and the patient is an Asian patient.

In one embodiment, 320 mg of capivasertib is orally administered to the patient twice daily; 4 days on, 3 days off; 600 mg of ribociclib is orally administered to the patient once daily for 21 consecutive days followed by 7 days off; 500 mg of fulvestrant is intramuscularly administered to the patient once every 28 days, and optionally an additional 500 mg dose of fulvestrant is intramuscularly administered to the patient two weeks after the initial dose.

In one embodiment, the combination therapy is administered to the patient over a 28-day cycle in which 320 mg of capivasertib is orally administered to the patient twice daily on days 1 to 4, 8 to 11, 15 to 18, and 22 to 25; 600 mg of ribociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

In one embodiment, the combination therapy is administered to the patient over a 28-day cycle in which 400 mg of capivasertib is orally administered to the patient twice daily on days 1 to 4, 8 to 11, 15 to 18, and 22 to 25; 400 mg of ribociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

In one embodiment, the combination therapy is administered to the patient over a 28-day cycle in which 400 mg of capivasertib is orally administered to the patient twice daily on days 1 to 4, 8 to 11, 15 to 18, and 22 to 25; 600 mg of ribociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

In one embodiment, the combination therapy is administered to the patient over a 28-day cycle in which 400 mg of capivasertib is orally administered to the patient twice daily on days 1 to 4, 8 to 11, 15 to 18, and 22 to 25; 150 mg of abemaciclib is orally administered to the patient twice daily on days 1 to 28; and 500 mg of fulvestrant is intramuscularly administered to the patient once daily on days 1 and 15 of the first 28-day cycle, and once on day 1 of the 28-day cycle thereafter.

### Patient Characteristics

In one embodiment, the patient has previously received endocrine therapy. In one embodiment, the endocrine therapy is tamoxifen or an aromatase inhibitor. In one embodiment, the aromatase inhibitor is selected from anastrozole (sold under the brand name ARIMIDEX, among others), exemestane (sold under the brand name AROMASIN, among others) and letrozole (sold under the brand name FEMARA, among others). In one embodiment, the endocrine therapy is a SERD, such as fulvestrant or an oral SERD. Examples of oral SERDs include elacestrant, amcenestrant, camizestrant, giredestrant, imlunestrant and rintodestrant. In one embodiment, the endocrine therapy is selected from tamoxifen, an aromatase inhibitor and a SERD. In one embodiment, the endocrine therapy is selected from tamoxifen, an aromatase inhibitor and an oral SERD.

In one embodiment, the patient has previously received adjuvant endocrine therapy. In one embodiment, the adjuvant endocrine therapy is tamoxifen or an aromatase inhibitor. In one embodiment, the aromatase inhibitor is selected from anastrozole, exemestane and letrozole. In one embodiment, the adjuvant endocrine therapy is an oral SERD. Examples of oral SERDs include elacestrant, amcenestrant, camizestrant, giredestrant, imlunestrant and rintodestrant. In one embodiment, the adjuvant endocrine therapy is selected from tamoxifen, an aromatase inhibitor and an oral SERD.

In one embodiment, the patient's breast cancer has recurred or progressed following endocrine therapy. In one embodiment, the patient's breast cancer has progressed following endocrine therapy. In one embodiment, the patient's breast cancer has recurred or progressed following adjuvant endocrine therapy. In one embodiment, the patient's breast cancer has progressed following adjuvant endocrine therapy. In one embodiment, the patient's breast cancer has recurred or progressed during, or within twelve months of the patient completing, adjuvant endocrine therapy. In one embodiment, the combination therapy is administered to the patient as a first-line therapy for hormone receptor-positive (HR+), HER2-negative locally advanced or metastatic breast cancer following recurrence or progression during, or within twelve months of the patient completing, adjuvant endocrine therapy.

In one embodiment, the patient's breast cancer has progressed during endocrine therapy for locally advanced or metastatic breast cancer. In one embodiment, the combination therapy is administered to the patient as a second-line therapy for hormone receptor-positive (HR+), HER2-negative locally advanced or metastatic breast cancer following progression during endocrine therapy for locally advanced or metastatic breast cancer.

In one embodiment, the patient has previously received therapy with fulvestrant. In one embodiment, the patient has previously received fulvestrant for advanced breast cancer. In one embodiment, the patient's breast cancer has progressed during endocrine therapy with fulvestrant for locally advanced or metastatic breast cancer. In one embodiment, the combination therapy is administered to the patient as a second-line therapy for hormone receptor-positive (HR+), HER2-negative locally advanced or metastatic breast cancer following progression during endocrine therapy with fulvestrant for locally advanced or metastatic breast cancer.

In one embodiment, the patient has previously received chemotherapy for advanced breast cancer. In one embodiment, the patient has previously received chemotherapy for locally advanced breast cancer. In one embodiment, the patient has previously received chemotherapy for metastatic breast cancer. In one embodiment, the patient has previously received no more than 2 lines of chemotherapy. In one embodiment, the patient has previously received no more than 1 line of chemotherapy. As used herein, the term “chemotherapy” refers to cytotoxic chemotherapy.

In one embodiment, the patient has previously received a CDK4/6 inhibitor as an adjuvant therapy. In one embodiment, the patient has previously received palbociclib, ribociclib and/or abemaciclib as an adjuvant therapy. In one embodiment, the combination therapy is not administered to the patient until 12 months have elapsed from completion of the prior adjuvant therapy with the CDK4/6 inhibitor.

In one embodiment, the patient has not previously received therapy with a SERD, for example fulvestrant, for locally advanced or metastatic breast cancer.

In one embodiment, the patient has not previously received therapy with a CDK4/6 inhibitor. In one embodiment, the patient has not previously received a CDK4/6 inhibitor as a therapy for locally advanced or metastatic breast cancer. In one embodiment, the patient has not previously received palbociclib, ribociclib and/or abemaciclib as a therapy for locally advanced or metastatic breast cancer.

In one embodiment, the patient's tumour cells comprise one or mutations in the AKT1, PIK3CA and/or PTEN genes. In one embodiment, the patient's tumour cells comprise one or mutations in the AKT1, PIK3CA and/or PTEN genes detectable by an approved test, for example the FOUNDATIONONE E CDX (FDA PMA No. P170019/S048, https://www.accessdata.fda.gov/scripts/cdrh/cfdocs/cfpma/pma.cfm?id=P170019S048).

In one embodiment, the patient's tumour cells comprise one or more of the following mutations:
i. E17K in the AKT1 gene;
ii. Any of the mutations in the PIK3CA gene listed in Table 2; and
iii. Any of the mutations in the PTEN gene listed in Table 3 or in Table 4.

In one embodiment, the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

In one embodiment, the patient's tumour cells comprise one or more of the alterations described in Table A.

In one embodiment, the patient's tumour cells do not comprise any of the alterations described in Table A.

The human wild-type PIK3CA, AKT1 and PTEN genes are identified in Table 1.

TABLE 1

| Gene | Full Name | Ensemble Transcript | Refseq |
|---|---|---|---|
| PIK3CA | phosphatidylinositol-4,5- | ENST00000263967 | NM_006218.2 |
| | bisphosphate 3-kinase, catalytic | | |
| | subunit alpha | | |
| AKT1 | AKT serine/threonine kinase 1 | ENST00000555528.5 | NM_005163 |
| PTEN | phosphatase and tensin homolog | ENST00000371953 | NM_000314.4 |

As PIK3CA and AKT1 are oncogenes, mutations that result in activation of the protein affect the PIK3CA/AKT1/PTEN pathway. A list of qualifying mutations in AKT1 and PIK3CA genes is provided in Table 2.

TABLE 2

| Gene | Mutation | Exon | Comments |
|---|---|---|---|
| AKT1 | E17K | 2 | Hotspot mutation |
| PIK3CA | R88Q | 1 | PI3K-ABD (p85) binding domain |
| PIK3CA | N345K | 4 | |
| PIK3CA | C402R | 7 | |
| PIK3CA | E542K | 9 | Helical domain |
| PIK3CA | E545A | 9 | Helical domain |
| PIK3CA | E545D | 9 | Helical domain |
| PIK3CA | E545Q | 9 | Helical domain |
| PIK3CA | E545K | 9 | Helical domain |
| PIK3CA | E545G | 9 | Helical domain |
| PIK3CA | Q546E | 9 | Helical domain |
| PIK3CA | Q546K | 9 | Helical domain |
| PIK3CA | Q546R | 9 | Helical domain |
| PIK3CA | Q546P | 9 | Helical domain |
| PIK3CA | M1043V | 20 | PI3_PI4_Kinase domain |
| PIK3CA | M1043I | 20 | PI3_PI4_Kinase domain |
| PIK3CA | H1047Y | 20 | PI3_PI4_Kinase domain |
| PIK3CA | H1047R | 20 | PI3_PI4_Kinase domain |
| PIK3CA | H1047L | 20 | PI3_PI4_Kinase domain |
| PIK3CA | G1049R | 20 | PI3_PI4_Kinase domain |

PTEN is a tumour suppressor gene, therefore gene alterations that result in loss of a functional protein affect the PIK3CA/AKT1/PTEN pathway. Details of the criteria to identify qualifying alterations in the PTEN gene are provided in Table 3, and further specific qualifying missense mutations are provided in Table 4.

TABLE 3

| Inclusion criteria for | |
|---|---|
| deleterious PTEN | |
| alterations | Additional information |
| Nonsense mutation | Any nonsense mutation leads to interrupted translation/ |
| i.e. creates a STOP codon | nonsense-mediated decay (NMD). |
| Frameshift Mutation | Any frameshift mutation usually leads to interrupted |
| i.e. insertion or deletion | translation/NMD. |
| causing a frameshift in the | |
| coding sequence | |
| Splicing Mutation | Mutations or deletion of any of the 4 bases that immediately |
| | flank coding exons (i.e. −2, −1, +1 or +2 to a coding exon) |
| | are expected to lead to incorrect splicing, which likely leads |
| | to a non-functional protein. |
| Start Codon Mutation | Any change to the recognised start codon (ATG/Methionine |
| | 1) for the main transcript should lead to loss of protein |
| | translation. |
| Deep deletion | Partial or entire PTEN homozygous gene deletion qualifies. |
| Intragenic Rearrangement | Any large-scale change within the PTEN gene considered |
| i.e. any rearrangement of | to be disruptive, including rearrangements and fusion (with |
| the PTEN locus clearly | another gene) Note: duplication, gain or amplification do |
| disruptive to the gene. | not qualify. |

TABLE 4

| | Protein | | |
|---|---|---|---|
| Gene | change | exon | Additional information |
| PTEN | C124R | 5 | Associated with Cowden Syndrome; in |
| | | | phosphatase p-loop |
| PTEN | C124S | 5 | In phosphatase p-loop |
| PTEN | G129E | 5 | In phosphatase p-loop |
| PTEN | G129V | 5 | In phosphatase p-loop |
| PTEN | G129R | 5 | In phosphatase p-loop |
| PTEN | R130Q | 5 | Associated with Cowden Syndrome, commonly |
| | | | mutation position across multiple tumour types |
| PTEN | R130G | 5 | Associated with Cowden Syndrome, commonly |
| | | | mutation position across multiple tumour types |
| PTEN | R130L | 5 | Associated with Cowden Syndrome, commonly |
| | | | mutation position across multiple tumour types |
| PTEN | R130P | 5 | Associated with Cowden Syndrome, commonly |
| | | | mutation position across multiple tumour types |
| PTEN | C136R | 5 | Associated with hereditary cancer-predisposing |
| | | | syndrome, associated with hamartoma tumour |
| | | | syndrome |
| PTEN | C136Y | 5 | Associated with Cowden Syndrome, associated |
| | | | with hereditary cancer-predisposing syndrome |
| PTEN | S170R | 6 | in Tl-loop |
| PTEN | R173C | 6 | Located at the phosphatase-C2 domain |

The sample obtained from the patient may be any sample type that contains breast tumour genomic material (e.g. tissue, blood, plasma or cell-free DNA). In one embodiment, the sample is a breast tumour tissue sample. In one embodiment, the sample is a sample containing ctDNA.

There are a variety of methods which are routinely used in the art to detect genetic mutations, and any suitable method can be used.

Next-generation sequencing (NGS) technologies can detect hundreds of alterations across multiple genes in a single test, and as the skilled person will be aware, NGS can be used to define tumour biomarker status. A single NGS assay can sensitively detect activating PIK3CA mutations and AKT1 mutations across their entire gene structures, as well as PTEN alterations and gene deletion.

In one embodiment, NGS is used to detect the presence or absence of any of the mutations detailed in Tables 2-4 in a sample containing tumour cells obtained from the patient. In one embodiment, the sample is a breast tumour tissue sample.

Commercially available NGS technologies include the FoundationOne®CDx (F1CDx) NGS Clinical Trial Assay from Foundation Medicine, Cambridge, MA, USA), which can be used to detect single-nucleotide variations, insertion and deletion alterations, and copy number alterations in DNA isolated from formalin-fixed paraffin-embedded tumour tissue specimens. The GuardantOMNI™ (Guardant Health, Redwood City, CA, USA) detects single-nucleotide variations, insertion and deletion alterations, copy number alterations, or fusions in 500 genes, including PIK3CA, AKT1, and PTEN alterations, using NGS of cfDNA extracted from plasma samples.

In one embodiment, the patient exhibits a molecular response to the combination therapy after one 28-day cycle of treatment. In one embodiment, the ctDNA burden of the patient is reduced by more than 50% after one 28-day cycle of treatment with the combination therapy.

In one embodiment, the PFS of the patient treated with the combination therapy is increased by 3 or more months, 4 or more months, 5 or more months, 6 or more months, 7 or more months, 8 or more months, 9 or more months, 10 or more months, 11 or more months, or 12 or more months compared to treatment with fulvestrant and a CDK4/6 inhibitor without capivasertib.

PFS is defined as time from date of first dose until progression per RECIST v1.1 criteria or death due to any cause.

In one embodiment, the PFS of the patient treated with the combination therapy is increased by 3 or more months, 4 or more months, 5 or more months, 6 or more months, 7 or more months, 8 or more months, 9 or more months, 10 or more months, 11 or more months, or 12 or more months compared to treatment with fulvestrant and a CDK4/6 inhibitor without capivasertib, and the patient's tumour cells comprise one or more of the following mutations:
i. E17K in the AKT1 gene;
ii. Any of the mutations in the PIK3CA gene listed in Table 2; and
iii. Any of the mutations in the PTEN gene listed in Table 3 or in Table 4.

In one embodiment, the PFS of the patient treated with the combination therapy is increased by 3 or more months, 4 or more months, 5 or more months, 6 or more months, 7 or more months, 8 or more months, 9 or more months, 10 or more months, 11 or more months, or 12 or more months compared to treatment with fulvestrant and a CDK4/6 inhibitor without capivasertib, the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

In one embodiment, the PFS2 of the patient treated with the combination therapy is increased by 3 or more months, 4 or more months, 5 or more months, 6 or more months, 7 or more months, 8 or more months, 9 or more months, 10 or more months, 11 or more months, or 12 or more months compared to treatment with fulvestrant and a CDK4/6 inhibitor without capivasertib.

PFS2 is defined as time from date of first dose to the earliest of the progression event (following the initial progression), subsequent to first subsequent therapy or death.

In one embodiment, the OS of the patient treated with the combination therapy is increased by 3 or more months, 4 or more months, 5 or more months, 6 or more months, 7 or more months, 8 or more months, 9 or more months, 10 or more months, 11 or more months, or 12 or more months compared to treatment with fulvestrant and a CDK4/6 inhibitor without capivasertib.

OS is defined as time from date of first dose until the date of death due to any cause.

In one embodiment, the combination therapy described herein provides an ORR of 40% or more, 45% or more, 50% or more, 55% or more, 60% or more, 65% or more, 70% or more, 75% or more, 80% or more, 85% or more, 90% or more, or 95% or more.

ORR is defined as the proportion of participants who have a complete response (CR) or partial response (PR) per RECIST v1.1.

In one embodiment, the combination therapy described herein provides a CBR at 24 weeks of 40% or more, 45% or more, 50% or more, 55% or more, 60% or more, 65% or more, 70% or more, 75% or more, 80% or more, 85% or more, 90% or more, or 95% or more.

CBR at 24 weeks is defined as the percentage of participants who have a CR or PR or who have SD per RECIST v1.1 for at least 23 weeks after date of first dose.

In some embodiments, the patient is a post-menopausal woman (defined by having at least one of the following criteria: (1) age ≥60 years, (2) prior bilateral oophorectomy, and (3) age <60 years with amenorrhea for ≥12 months (consecutive) and follicle stimulating hormone (FSH) and oestradiol at screening within postmenopausal range per local laboratory). In some embodiments, the patient is a premenopausal or perimenopausal woman (i.e. a woman not meeting the criteria for a post-menopausal woman) and is concomitantly treated with a luteinizing hormone-releasing hormone (LHRH) agonist such as leuprolide or goserelin. In some embodiments, the patient is male and is concomitantly treated with a luteinizing hormone-releasing hormone (LHRH) agonist such as leuprolide or goserelin.

In one embodiment, the patient is ≥18 years old.

### Combination Therapies for Use

Further specific embodiments are disclosed below:
Embodiment A1. A combination therapy for use in treating a patient having hormone receptor-positive (HR+), HER2-negative locally advanced or metastatic breast cancer, wherein the combination therapy comprises capivasertib, a CDK4/6 inhibitor and fulvestrant.
Embodiment A2. The combination therapy for use of Embodiment A1, wherein capivasertib is administered to the patient at an amount of 400 mg twice daily.
Embodiment A3. The combination therapy for use of Embodiment A1 or A2, wherein the CDK4/6 inhibitor is selected from palbociclib, ribociclib and abemaciclib.
Embodiment A4. The combination therapy for use of Embodiment A1 or A2, wherein the CDK4/6 inhibitor is palbociclib.
Embodiment A5. The combination therapy for use of Embodiment A4, wherein palbociclib is administered to the patient at an amount of 125 mg once daily.
Embodiment A6. The combination therapy for use of Embodiment A4, wherein palbociclib is administered to the patient at an amount of 100 mg once daily.
Embodiment A7. The combination therapy for use of Embodiment A1, wherein capivasertib is administered to the patient at an amount of 320 mg twice daily.
Embodiment A8. The combination therapy for use of any one of Embodiments A1, A2 and A7, wherein the CDK4/6 inhibitor is ribociclib.
Embodiment A9. The combination therapy for use of Embodiment A8, wherein ribociclib is administered to the patient at an amount of 600 mg once daily.
Embodiment A10. The combination therapy for use of any one of Embodiments A1 to A9, wherein fulvestrant is administered to the patient at an amount of 500 mg once monthly.
Embodiment A11. The combination therapy for use of Embodiment A1, wherein the combination therapy is administered to the patient over a 28-day cycle in which 400 mg of capivasertib is orally administered to the patient twice daily on days 1 to 4, 8 to 11, 15 to 18, and 22 to 25; 125 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.
Embodiment A12. The combination therapy for use of Embodiment A1, wherein the combination therapy is administered to the patient over a 28-day cycle in which 400 mg of capivasertib is orally administered to the patient twice daily on days 1 to 4, 8 to 11, 15 to 18, and 22 to 25; 100 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.
Embodiment A13. The combination therapy for use of Embodiment A6 or A12, wherein the patient is a Japanese patient.
Embodiment A14. The combination therapy for use of Embodiment A6 or A12, wherein the patient is an Asian patient.
Embodiment A15. The combination therapy for use of Embodiment A1, wherein the combination therapy is administered to the patient over a 28-day cycle in which 320 mg of capivasertib is orally administered to the patient twice daily on days 1 to 4, 8 to 11, 15 to 18, and 22 to 25; 600 mg of ribociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.
Embodiment A16. The combination therapy for use of any one of Embodiments A1 to A15, wherein the patient has previously received endocrine therapy.
Embodiment A17. The combination therapy for use of any one of Embodiments A1 to A16, wherein the patient's breast cancer has recurred or progressed during, or within twelve months of the patient completing, adjuvant endocrine therapy.
Embodiment A18. The combination therapy for use of any one of Embodiments A1 to A16, wherein the patient's breast cancer has progressed during endocrine therapy for locally advanced or metastatic breast cancer.
Embodiment A19. The combination therapy for use of Embodiment A16, A17 or A18, wherein the endocrine therapy is tamoxifen or an aromatase inhibitor.
Embodiment A20. The combination therapy for use of any one of Embodiments A1 to A19, wherein the patient has previously received an oral SERD as an adjuvant therapy.
Embodiment A21. The combination therapy for use of any one of Embodiments A1 to A20, wherein the patient has previously received therapy with fulvestrant.
Embodiment A22. The combination therapy for use of any one of Embodiments A1 to A21, wherein the patient has previously received chemotherapy for advanced breast cancer.
Embodiment A23. The combination therapy for use of any one of Embodiments A1 to A22, wherein the patient has previously received a CDK4/6 inhibitor as an adjuvant therapy.
Embodiment A24. The combination therapy for use of any one of Embodiments A1 to A23, wherein the patient has not previously received a CDK4/6 inhibitor as a therapy for locally advanced or metastatic breast cancer.
Embodiment A25. The combination therapy for use of any one of Embodiments A1 to A24, wherein the patient's tumour cells comprise one or more of the following mutations:
   i. E17K in the AKT1 gene;
   ii. Any of the mutations in the PIK3CA gene listed in Table 2; and
   iii. Any of the mutations in the PTEN gene listed in Table 3 or in Table 4.
Embodiment A26. The combination therapy for use of any one of Embodiments A1 to A24, wherein the patient's tumour cells do not comprise any of the following mutations:
   i. E17K in the AKT1 gene;
   ii. Any of the mutations in the PIK3CA gene listed in Table 2; and
   iii. Any of the mutations in the PTEN gene listed in Table 3 or in Table 4.
Embodiment A27. The combination therapy for use of any one of Embodiments A1 to A26, wherein the patient exhibits a molecular response to the combination therapy after one 28-day cycle of treatment.
Embodiment A28. The combination therapy for use of any one of Embodiments A1 to A26, wherein the ctDNA burden of the patient is reduced by more than 50% after one 28-day cycle of treatment with the combination therapy.
Embodiment A29. The combination therapy for use of any one of Embodiments A1 to A28, wherein the combination therapy provides an ORR of 40% or more.
Embodiment A30. The combination therapy for use of any one of Embodiments A1 to A29, wherein the combination therapy provides a CBR at 24 weeks of 40% or more.

### Pharmaceutically Acceptable Salts

In an embodiment, the active ingredients in the combination therapies described herein may be replaced with pharmaceutically acceptable salts of such active ingredients. In one embodiment, capivasertib may be replaced with a pharmaceutically acceptable salt of capivasertib. In one embodiment, palbociclib may be replaced with a pharmaceutically acceptable salt of palbociclib. In one embodiment, ribociclib may be replaced with a pharmaceutically acceptable salt of ribociclib. In one embodiment, abemaciclib may be replaced with a pharmaceutically acceptable salt of abemaciclib. In each case, the amount of pharmaceutically acceptable salt of an active ingredient utilised in the combination therapy is chosen such that it provides the same amount of the active ingredient in its free form.

The term “pharmaceutically acceptable” is used to specify that a salt is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich: Wiley-VCH/VHCA, 2002, which is incorporated by reference herein in its entirety.

### EXAMPLES

### Example 1: CAPItello-292 Clinical Study (ClinicalTrials.Gov Identifier: NCT04862663, Incorporated by Reference Herein in its Entirety)

Protocol Title: A Phase Ib/III Randomised Study of Capivasertib plus CDK4/6 Inhibitors and Fulvestrant versus Placebo plus CDK4/6 Inhibitors and Fulvestrant in Hormone Receptor-Positive and Human Epidermal Growth Factor Receptor 2-Negative Locally Advanced, Unresectable or Metastatic Breast Cancer (CAPItello-292)

### Overall Design

This is a Phase Ib/III, multicentre study of capivasertib plus CDK4/6i (palbociclib, ribociclib or abemaciclib) and fulvestrant, for the treatment of participants with locally advanced (inoperable) or metastatic HR+/HER2− breast cancer following either recurrence or progression on, or after (neo) adjuvant ET. The study comprises two parts (Phase Ib and Phase III); the recommended Phase III doses (RP3Ds) of capivasertib and CDK4/6i in combination with a fixed dose of fulvestrant will be determined in the open-label, dose finding Phase Ib part. The efficacy and safety of capivasertib plus palbociclib and fulvestrant will be compared to placebo plus palbociclib and fulvestrant in the randomised, open-label Phase III part. The efficacy and safety of capivasertib plus ribociclib and fulvestrant will also be compared to placebo plus ribociclib and fulvestrant in the randomised, open-label Phase III part. The efficacy and safety of the capivasertib arm will be compared to the control arm in the randomised, open-label Phase III part.

The Phase Ib part of the study consists of an open-label, 3-arm dose finding phase to determine the safe and well tolerated doses and schedules (RP3D) of capivasertib plus CDK4/6i (palbociclib, ribociclib, or abemaciclib) and fulvestrant (the latter administered at fixed dose and schedule, as per label) as triplet combinations in participants with ABC. The maximum tolerated doses (MTD) may also be determined.

The Phase III part is an open-label, randomised study assessing the efficacy of the capivasertib arm (capivasertib plus investigator's choice of CDK4/6i [palbociclib or ribociclib] and fulvestrant) versus the control arm (investigator's choice of CDK4/6i [palbociclib or ribociclib] and fulvestrant) for the treatment of patients with locally advanced (inoperable) or metastatic HR+/HER2− breast cancer following recurrence or progression on or after endocrine therapy. The Phase III doses of capivasertib, palbociclib, and fulvestrant combination and capivasertib, ribociclib, and fulvestrant combination were identified in the Phase Ib part of this study, based on the safety and tolerability data that were reviewed and endorsed by an external Safety Review Committee (SRC).

HR+/HER2− breast cancer must be histologically determined preferably from the most recently collected pre-randomisation tumour sample (primary or metastatic), as per the American Society of Clinical Oncology-College of American Pathologists guideline recommendations (Hammond M E H et al., J Clin Oncol 2010, 28 (16), 2784-95; Erratum in: J Clin Oncol. 2010, 28 (21), 3543; Wolff A C et al., Arch Pathol Lab Med 2018, 142 (11), 1364-82). To fulfil the requirement of HR+ disease, a breast cancer must express ER with or without co-expression of progesterone receptor.

Participants must have radiologic evidence of recurrence or progression and an Eastern Cooperative Oncology Group (ECOG) Performance Status of 0 or 1.

Adequate haematology, glucose metabolism, renal and liver function tests are required at screening; patients with type 2 diabetes are eligible if glycated haemoglobin (HbA1c) at screening is less than 8.0% (63.9 mmol/mol), and if they do not require insulin treatment.

A formalin-fixed paraffin embedded (FFPE) tumour tissue block (strongly preferred) or freshly-cut unstained, serial slides preferably from the most recently collected pre-randomisation tumour tissue will be required from all participants [20 (minimum 15) for Phase Ib, 30 (minimum 20) for Phase III].

For the Phase Ib part of the study, participants must be eligible for fulvestrant therapy and at least one of the following: palbociclib, ribociclib, or abemaciclib, as per local investigator assessment. Participants who received treatment with at least one prior endocrine therapy (alone or in combination) for ABC and a maximum of 2 lines prior chemotherapy (for global participants) or up to 1 prior line of chemotherapy (for Japanese participants) are eligible.

For the Phase III part, participants must:
Be eligible for fulvestrant and at least one out of palbociclib or ribociclib (depending on the available CDK4/6i options at time of enrolment), as per local investigator assessment.
Have radiologic evidence of recurrence or progression while on, or within 12 months of the end of (neo) adjuvant endocrine treatment (tamoxifen, AI, or oral SERD). Note: in this clinical study protocol, (neo) adjuvant treatment is defined as neoadjuvant and/or adjuvant treatment.
Have measurable lesion(s) according to Response Evaluation Criteria in Solid Tumours version 1.1 (RECIST v1.1) or, in the absence of measurable disease, lytic or mixed bone lesions that can be assessed by computed tomography (CT) or magnetic resonance imaging (MRI).

Eligible participants may have received up to 1 line of chemotherapy in the metastatic setting.

Participants with previous or concurrent treatment with AKT, PI3K, and mTOR inhibitors, ET, or CDK4/6i in the metastatic setting are not eligible for the Phase III study.

TABLE 5
Objectives and Endpoints for the Phase Ib Part of the Study

| | |
|---|---|
| Objectives | Endpoints |

Primary

| | |
|---|---|
| To assess the safety and tolerability of | Safety and tolerability will be evaluated |
| capivasertib plus CDK4/6i (palbociclib, | in terms of DLTs, AEs/SAEs, vital signs, |
| ribociclib or abemaciclib) and | clinical chemistry/haematology/glucose |
| fulvestrant when dosed concomitantly | metabolism parameters, and ECG |
| in participants with advanced breast | parameters. |
| cancer. | The measures of interest are subject |
| To confirm the RP3D and/or maximum | incidence rates, absolute values and |
| tolerated dose of the triplets. | change from baseline over time. |

Secondary

| | |
|---|---|
| To compare the pharmacokinetics of | Palbociclib PK parameters: Cₘₐₓ, |
| palbociclib, ribociclib or abemaciclib | AUC_{0-72 h} (Cycle 0), AUC_{0-24 h} (Cycle 1), |
| dosed alone and in combination with | Cₘᵢₙ (Cycle 1) |
| capivasertib and fulvestrant. | Ribociclib PK parameters: Cₘₐₓ, AUC₀₋ₜ |
| | (Cycle 0), AUC_{0-24 h} (Cycle 1), Cₘᵢₙ |
| | (Cycle 1) |
| | Abemaciclib PK parameters: Cₘₐₓ, |
| | AUC₀₋ₜ (Cycle 0), AUC_{0-12 h} (Cycle 1), |
| | Cₘᵢₙ (Cycle 1) |
| | Plasma PK parameters derived from a |
| | population PK model, as permitted by |
| | the data |
| To assess the pharmacokinetics of | Capivasertib PK parameters: Cₘₐₓ, |
| capivasertib in combination with CDK4/6i | AUC_{0-12 h}, Cₘᵢₙ |
| (palbociclib, ribociclib, or abemaciclib) | Plasma PK parameters derived from a |
| and fulvestrant. | population PK model, as permitted by |
| | the data |
| To estimate the effectiveness of | ORR is defined as the proportion of |
| capivasertib plus CDK4/6i (palbociclib, | participants with measurable disease at |
| ribociclib or abemaciclib) and fulvestrant | baseline who have a confirmed complete |
| when dosed concomitantly by | response (CR) or confirmed partial |
| assessment of objective response rate | response (PR), as determined by the |
| (ORR), Clinical Benefit Rate (CBR), | investigator at local site per RECIST v1.1. |
| Duration of Response (DoR) for | CBR at 24 weeks is defined as the |
| participants with measurable disease and | percentage of participants who have a CR |
| progression free survival (PFS) in all | or PR or who have SD per RECIST v1.1 |
| participants. | as assessed by the investigator at local |
| | site for at least 23 weeks after date of |
| | first dose. |
| | DoR will be defined as the time from the |
| | date of first documented confirmed |
| | response until date of documented |
| | progression per RECIST v1.1 as |
| | assessed by the investigator at local site |
| | or death due to any cause. |
| | PFS is defined as time from date of |
| | first dose until progression per RECIST |
| | v1.1. as assessed by the investigator at |
| | local site or death due to any cause. |

Safety

| | |
|---|---|
| As described above | As described above |

TABLE 6
Objectives and Endpoints for the Phase III Part of the Study

| | |
|---|---|
| Objectives | Endpoint/variable |

Primary

| | |
|---|---|
| To demonstrate the superiority of the | PFS is defined as time from |
| capivasertib arm relative to control arm by | randomisation until progression per |
| assessment of PFS in participants with | RECIST v1.1 as assessed by Blinded |
| HR+/HER2− locally advanced or | Independent Central Review (BICR), or |
| metastatic breast cancer (overall | death due to any cause. The comparison |
| population). | will include all randomised participants, |
| | as randomised, regardless of whether the |
| | participant withdraws from randomised |
| | therapy, receives another anti-cancer |
| | therapy or clinically progresses prior to |
| | RECIST v1.1 progression. However, if the |
| | participant progresses or dies |
| | immediately after two or more |
| | consecutive missed visits, the participant |
| | will be censored at the time of the latest |
| | evaluable assessment prior to the two |
| | missed visits. |
| | The measure of interest is the Hazard |
| | Ratio (HR) of PFS. |
| To demonstrate the superiority of the | PFS defined as above. |
| capivasertib arm relative to control arm by | The comparison will include all |
| assessment of PFS in participants with | participants with detected |
| HR+/HER2− locally advanced or | PIK3CA/AKT1/PTEN |
| metastatic breast cancer and gene | alterations/mutations at baseline by |
| alterations detected in PIK3CA, AKT1, | tumour tissue testing (any participant with |
| and/or PTEN (PIK3CA/AKT1/PTEN- | no PIK3CA/AKT1/PTEN result will be |
| altered population). | excluded from analysis in the altered or |
| | confirmed non-altered subgroups). |
| | The measure of interest is the HR of PFS. |
| To demonstrate the superiority of the | PFS defined as above. |
| capivasertib arm relative to control arm by | The comparison will include all |
| assessment of PFS in participants with | participants confirmed to be without |
| HR+/HER2− locally advanced or | PIK3CA/AKT1/PTEN |
| metastatic breast cancer and without | alterations/mutations at baseline by |
| gene alterations in PIK3CA, AKT1, and/or | tumour tissue testing (any participant with |
| PTEN (confirmed non-altered population). | no PIK3CA/AKT1/PTEN result will be |
| | excluded from analysis in the altered or |
| | confirmed non-altered subgroups). |
| | The measure of interest is the HR of PFS. |

Secondary

| | |
|---|---|
| To demonstrate the effectiveness of the | OS is defined as time from randomisation |
| capivasertib arm relative to control arm by | until the date of death due to any cause. |
| assessment of overall survival (OS) in | The comparison will include all |
| participants with HR+/HER2− locally | randomised participants, as randomised, |
| advanced or metastatic breast cancer | regardless of whether the participant |
| (overall population). | withdraws from therapy or receives |
| | another anti-cancer therapy. |
| | The measure of interest is the HR of OS. |
| To assess the effectiveness of the | OS as defined above. The comparison |
| capivasertib arm relative to control arm by | will include all participants with detected |
| assessment of OS in participants with | PIK3CA/AKT1/PTEN |
| HR+/HER2− locally advanced or | alterations/mutations at baseline by |
| metastatic breast cancer | tumour tissue testing (any participant with |
| (PIK3CA/AKT1/PTEN-altered population). | no PIK3CA/AKT1/PTEN result will be |
| | excluded from analysis in the altered or |
| | confirmed non-altered subgroups). |
| | The measure of interest is the HR of OS. |
| To assess the effectiveness of the | OS as defined above. The comparison |
| capivasertib arm relative to control arm by | will include all participants confirmed to |
| assessment of OS in participants with | be without PIK3CA/AKT1/PTEN |
| HR+/HER2− locally advanced or | alterations/mutations at baseline by |
| metastatic breast cancer (confirmed non- | tumour tissue testing (any participant with |
| altered population). | no PIK3CA/AKT1/PTEN result will be |
| | excluded from analysis in the altered or |
| | confirmed non-altered subgroups). |
| | The measure of interest is the HR of OS. |
| To assess the effectiveness of the | PFS2 defined as time from randomisation |
| capivasertib arm relative to control arm by | to the earliest of the progression event |
| assessment of PFS2 in in participants | (following the initial progression), after |
| with HR+/HER2− locally advanced or | first subsequent therapy, or death. The |
| metastatic breast cancer (overall | date of second progression will be |
| population). | recorded by the investigator in the eCRF |
| | and defined according to local |
| | standard clinical practice. |
| | The measure of interest is the HR of PFS2. |
| To demonstrate the effectiveness of the | ORR is defined as the proportion of |
| capivasertib arm relative to control arm by | participants who have a complete |
| assessment of ORR in participants with | response (CR) or partial response (PR), |
| HR+/HER2− locally advanced or | as determined by BICR per RECIST v1.1. |
| metastatic breast cancer (overall | The analysis will include all randomised |
| population). | participants as randomised with |
| | measurable disease at baseline. |
| | Data obtained from randomisation up until |
| | progression, or the last |
| | evaluable assessment in the absence of |
| | progression, will be included in the |
| | assessment of ORR, regardless of |
| | whether the participant |
| | withdraws from therapy. Participants who |
| | go off treatment without a response |
| | or progression, receive a subsequent |
| | therapy, and then respond will not be |
| | included as responders in the ORR. |
| | The measure of interest is in the odds |
| | ratio of ORR. |
| To assess the effectiveness of the | ORR as defined above. The comparison |
| capivasertib arm relative to control arm by | will include all participants with detected |
| assessment of ORR in participants with | PIK3CA/AKT1/PTEN |
| HR+/HER2− locally advanced or | alterations/mutations at baseline by |
| metastatic breast cancer | tumour tissue testing (any participant with |
| (PIK3CA/AKT1/PTEN-altered population). | no PIK3CA/AKT1/PTEN result will be |
| | excluded from analysis in the altered or |
| | confirmed non-altered subgroups). |
| | The measure of interest is the odds ratio |
| | of ORR. |
| To assess the effectiveness of the | ORR as defined above. The comparison |
| capivasertib arm relative to control arm by | will include all participants confirmed to |
| assessment of ORR in participants with | be without PIK3CA/AKT1/PTEN |
| HR+/HER2− locally advanced or | alterations/mutations at baseline by |
| metastatic breast cancer (confirmed non- | tumour tissue testing (any participant with |
| altered population). | no PIK3CA/AKT1/PTEN result will be |
| | excluded from analysis in the altered or |
| | confirmed non-altered subgroups). |
| | The measure of interest is the odds ratio |
| | of ORR. |
| To assess the effectiveness of the | DoR is defined as the time from the date |
| capivasertib arm relative to control arm by | of first documented response (PR or CR) |
| assessment of DoR in participants with | until the date of documented progression |
| HR+/HER2− locally advanced or | per RECIST v1.1 as assessed by BICR, |
| metastatic breast cancer (overall | or death due to any cause. |
| population). | The analysis will include all randomised |
| | participants as randomised with |
| | measurable disease at baseline who have |
| | a response, regardless of whether the |
| | participant subsequently withdraws from |
| | therapy, receives another anti-cancer |
| | therapy or clinically progresses prior to |
| | RECIST v1.1 progression. |
| | The measure of interest is the median |
| | DoR in each treatment group based on |
| | KM methods. |
| To assess the effectiveness of the | CBR at 24 weeks is defined as the |
| capivasertib arm relative to control arm by | percentage of participants who have a CR |
| assessment of CBR in participants with | or PR or who have SD per RECIST v1.1 |
| HR+/HER2− locally advanced or | as assessed by BICR for at least |
| metastatic breast cancer (overall | 23 weeks after randomisation. |
| population). | Data obtained from randomisation up until |
| | progression, or the last evaluable |
| | assessment in the absence of |
| | progression, will be included in the |
| | assessment of CBR, regardless of |
| | whether the participant withdraws from |
| | therapy. Participants who receive a |
| | subsequent therapy prior to week 24 will |
| | not be considered to have clinical benefit |
| | in the analysis. |
| | The analysis will include all randomised |
| | participants as randomised with lesions |
| | (either TL or NTLs) recorded at baseline. |
| | The measure of interest is the odds ratio |
| | of the CBR at 24 weeks. |
| To assess participant-reported physical | TTD of physical functioning as measured |
| functioning in participants with | by the physical functioning subscale of |
| HR+/HER2− locally advanced or | the EORTC QLQ-C30. |
| metastatic breast cancer (overall | The analysis will include all randomised |
| population) | participants. |
| | The measure of interest is the HR of TTD |
| | in the physical functioning subscale |
| | scores from the EORTC QLQ-C30. |
| To assess participant-reported Global | TTD of GHS/QoL as measured by the |
| Health Status (GHS)/quality of life (QoL) | GHS/QoL subscale of the EORTC QLQ-C30. |
| in participants with HR+/HER2− locally | The analysis will include all randomised |
| advanced or metastatic breast cancer | participants. |
| (overall population) | The measure of interest will be HR of TTD |
| | of the GHS/QoL subscale score from the |
| | EORTC QLQ-C30. |
| To describe participant-reported overall | Proportion of participants experiencing |
| side effect bother in participants in the | different levels of overall treatment |
| capivasertib arm relative to control arm | tolerability as measured by the PGI-TT. |
| (overall population) | The analysis will include all dosed |
| | participants. |
| | The measure of interest will be proportion |
| | of participants reporting different levels of |
| | overall treatment tolerability measured by |
| | the PGI-TT. |
| To evaluate the PK of capivasertib | Plasma concentration of capivasertib |
| administered in combination with | pre-dose (C_{trough}), and post-dose (C_{1 h}, |
| investigator's choice of CDK4/6i | C_{2 h}, and C_{4 h}) in the overall population |
| (palbociclib or ribociclib) and | (participants randomised to |
| fulvestrant in participants with | capivasertib arm) |
| HR+/HER2− locally advanced or | Plasma PK parameters derived from a |
| metastatic breast cancer | population PK model, as permitted by |
| (overall population) | the data |
| To evaluate the PK of palbociclib when | Plasma concentration of palbociclib pre- |
| administered in combination with | dose (C_{trough}) |
| fulvestrant with and without capivasertib | |
| in participants with HR+/HER2− locally | |
| advanced or metastatic breast cancer | |
| (overall population) | |

Safety

| | |
|---|---|
| To assess the safety and tolerability of | Safety and tolerability will be evaluated in |
| the capivasertib arm relative to control | terms of AEs/SAEs, vital signs, clinical |
| arm in participants with HR+/HER2− | chemistry/haematology/glucose |
| locally advanced or metastatic breast | metabolism parameters, and ECG parameters. |
| cancer (overall population). | The measures of interest are AE |
| | incidence rates, absolute values and |
| | change from baseline over time. |
| To assess the safety and tolerability of | Safety and tolerability will be evaluated in |
| the capivasertib arm relative to control | terms of AEs/SAEs, vital signs, clinical |
| arm in participants with HR+/HER2− | chemistry/haematology/glucose |
| locally advanced or metastatic breast | metabolism parameters, and ECG parameters. |
| cancer (PIK3CA/AKT1/PTEN-altered | The measures of interest are AE |
| population). | incidence rates, absolute values and |
| | change from baseline over time. |
| To assess the safety and tolerability of | Safety and tolerability will be evaluated in |
| the capivasertib arm relative to control | terms of AEs/SAEs, vital signs, clinical |
| arm in participants with HR+/HER2− | chemistry/haematology/glucose |
| locally advanced or metastatic breast | metabolism parameters, and ECG |
| cancer (confirmed non-altered | parameters. |
| population). | The measures of interest are AE |
| | incidence rates, absolute values and |
| | change from baseline over time. |

TABLE 7
Investigational Products, Dosage and Mode of Administration

| | | |
|---|---|---|
| Intervention Name | Capivasertib | Fulvestrant |
| Type | Drug | Drug |
| Dose Formulation | Film-coated tablet | Intramuscular injection |
| Unit Dose Strength(s) | 160 mg and 200 mg | 250 mg/5 mL solution for injection |
| Dosage Level(s) | Potential range 200 | 500 mg (2 injections) on Day 1 of |
| | mg, 320 mg, 400 mg | Weeks 1 and 3 of Cycle 1, and then |
| | Days 1 to 4 in each | on Day 1, Week 1 of each cycle |
| | week of a 28-day | thereafter |
| | treatment cycle | |
| Route of Administration | Oral | Intramuscular |

| | | | |
|---|---|---|---|
| Intervention Name | Palbociclib | Ribociclib | Abemaciclib |
| Type | Drug | Drug | Drug |
| Dose Formulation | Capsules or tablets | Film-coated tablet | Film-coated tablet |
| Unit Dose Strength(s) | 75, 100 and 125 mg | 200 mg tablets | 50, 100, 150, and |
| | capsules or tablets | | 200 mg tablets |
| Dosage Level(s) | Potential range 75 | Potential range 200 | Potential range 50 |
| | mg, 100 mg, 125 | mg, 400 mg, 600 | mg, 100 mg, 150 |
| | mg. Once daily for | mg. Once daily for | mg. Twice daily for |
| | 21 consecutive days | 21 consecutive days | 28 consecutive days |
| | followed by 7 days | followed by 7 days | to comprise a |
| | off treatment to | off treatment to | complete 28-day |
| | comprise a complete | comprise a complete | cycle |
| | 28-day cycle | 28-day cycle | |
| Route of Administration | Oral | Oral | Oral |

### Dosing

### Capivasertib Dosing Instructions

Where possible, all doses of capivasertib should be taken:
At approximately the same time each day, 12 hours apart.
In the Phase Ib palbociclib arm, capivasertib should be taken with food, but not directly after a heavy meal (which is defined as approximately 700-800 kcal).
In the Phase Ib ribociclib and abemaciclib arms and in the Phase III part of the study, capivasertib can be taken with or without food.
If vomiting occurs, a replacement dose should not be taken.

Should a participant miss a scheduled dosing time, the participant will be allowed to take the dose up to a maximum of 4 hours after the scheduled dose time. If greater than 4 hours after the scheduled dose time, the missed dose should not be taken and the participant should take their allotted dose at the next scheduled time. If a participant needs to take the dose earlier for whatever reason, the participant can take the dose up to 4 hours earlier than the scheduled dose time. The participant should make every reasonable effort to take the capivasertib tablet(s) on time.

### Fulvestrant Dosing Instructions

Formulation, preparation, and administration according to local prescribing information.

### CDK4/6i Dosing Instructions

Formulation, preparation, and administration according to local prescribing information.

### Intervention Groups and Duration:

In the open-label Phase Ib part of the study:
The initial palbociclib cohort will receive capivasertib (320 mg oral twice daily, 4 days on and 3 days off for 4 weeks), palbociclib (125 mg, oral once daily, dosed continually for 21 days out of 28 days), and fulvestrant (500 mg intramuscular injection [IM] monthly [Day 1 of a 28-day cycle] with a loading dose of 500 mg 2 weeks after the first dose).
The initial ribociclib cohort will explore the combination of capivasertib (400 mg oral twice daily, 4 days on and 3 days off for 4 weeks), ribociclib (400 mg, oral once daily, dosed continually for 21 days out of 28 days), and fulvestrant (500 mg IM monthly [Day 1 of a 28-day cycle] with a loading dose of 500 mg 2 weeks after the first dose).
The initial abemaciclib cohort will explore the combination of capivasertib (400 mg oral twice daily, 4 days on and 3 days off for 4 weeks), abemaciclib (100 mg, oral twice daily, 28 days out of 28 days), and fulvestrant (500 mg IM monthly [Day 1 of a 28-day cycle] with a loading dose of 500 mg 2 weeks after the first dose).

A recommendation regarding the dose level for each of the next CDK4/6i cohorts of evaluable participants will be made by a Safety Review Committee (SRC), guided by decision rules following the Keyboard design for drug combination trials (Pan H et al., Contemp Clin Trials. 2020, 92, 105972; Yan F et al., Clin Cancer Res. 2017, 23 (15), 3994-4003). Available doses for capivasertib, the CDK4/6 inhibitor, and fulvestrant are described in Table 7.

In the Phase III part, participants will be randomised in a 1:1 ratio to the capivasertib arm or control arm. In the capivasertib arm, participants will receive capivasertib (320 mg in combination with ribociclib and 400 mg in combination with palbociclib, oral twice daily, 4 days on and 3 days off for 4 weeks), and in both arms participants will receive palbociclib (125 mg [100 mg in Japan], oral once daily, 21 days out of a 28-day cycle) or ribociclib (600 mg, oral once daily, 21 days out of a 28-day cycle), and fulvestrant (500 mg IM monthly plus loading dose at Cycle 1). The Phase III doses of capivasertib, palbociclib, and fulvestrant combination and capivasertib, ribociclib, and fulvestrant combination were identified in the Phase Ib part of this study, based on the pharmacokinetic, safety and tolerability data that was reviewed and endorsed by an external SRC.

Randomisation will be stratified by the presence of liver metastases (liver metastases versus no liver metastases), sensitivity to prior hormonal (endocrine) therapy (≥24 months of adjuvant hormonal therapy prior to recurrence versus <24 months of adjuvant hormonal therapy prior to recurrence), investigator's choice of CDK4/6i (palbociclib versus ribociclib), and gene alteration status by circulating tumour DNA (ctDNA) testing (PIK3CA/AKT1/PTEN-altered versus confirmed non-altered [including unknowns]).

For both parts of the study, study intervention will be continued until disease progression unless there is evidence of unacceptable toxicity, or if the participant requests to stop the study treatment.

### Inclusion/Exclusion Criteria

Key inclusion criteria for both phases:
1. Adult females (pre- and post-menopausal), and adult males.
2. Histologically confirmed HR+/HER2− breast cancer determined from the most recent tumour sample (primary or metastatic) per the American Society of Clinical Oncology and College of American Pathologists guideline. To fulfil the requirement of HR+ disease, a breast cancer must express ER with or without co-expression of progesterone receptor.
3. Eligible for fulvestrant therapy and at least one of the following: palbociclib, ribociclib, or abemaciclib, as per local investigator assessment. Previous tolerance to specific CDK4/6 inhibitors and dose levels required.
4. Adequate organ and bone marrow functions.
5. Consent to provide a mandatory FFPE tumour sample.

Inclusion criteria only for phase III:
1. Previous treatment with an ET (tamoxifen, AI, or oral SERD) as a single agent or in combination, with radiological evidence of breast cancer recurrence or progression while on, or within 12 months of, completing a (neo) adjuvant ET regimen.

Key exclusion criteria for both phases:
1. History of another primary malignancy except for malignancy treated with curative intent with no known active disease ≥5 years before the first dose of study intervention and of low potential risk for recurrence.
2. Radiotherapy within 2 weeks prior to study treatment initiation.
3. Major surgery within 4 weeks of the first dose of study treatment.
4. Persistent toxicities (CTCAE Grade ≥1) caused by previous anticancer therapy, excluding alopecia. Participants with irreversible toxicity that is not reasonably expected to be exacerbated by study intervention may be included (eg, hearing loss or peripheral sensory neuropathy) after consultation with the AstraZeneca study physician.
5. Spinal cord compression, brain metastases or leptomeningeal metastases unless these lesions are definitively treated (eg. radiotherapy, surgery) and clinically stable off steroids for management of symptoms for at least 4 weeks prior to study treatment initiation.
6. Any of the following cardiac criteria at screening:
(a). Mean resting corrected QT interval (QTcF): (i) Palbociclib arm: QTcF ≥470 ms obtained from the average of 3 consecutive (triplicate) ECGs (ii) Phase Ib only: Ribociclib arm: QTcF ≥450 ms obtained from the average of 3 consecutive (triplicate) ECGs (iii) Phase Ib only: Abemaciclib arm: QTcF ≥470 ms obtained from the average of 3 consecutive (triplicate) ECGs (b). Any clinically important abnormalities in cardiac rhythm, conduction or morphology of resting ECG (eg, complete left bundle branch block, third-degree heart block) (c). Any factors that increase the risk of QTc prolongation or risk of arrhythmic events (d). Experience of any of the following procedures or conditions in the preceding 6 months: coronary artery bypass graft, angioplasty, vascular stent, myocardial infarction, unstable angina pectoris, congestive heart failure New York Heart Association (NYHA) grade ≥2 (e). Uncontrolled hypotension (f) uncontrolled hypertension (g). Cardiac ejection fraction outside institutional range of normal or <50% (whichever is higher)
7. Uncontrolled or high grade or symptomatic arrhythmia and atrial fibrillation.
8. Any of these clinically significant abnormalities of glucose metabolism at screening:
   a . . . diabetes mellitus type I or type II requiring insulin treatment
   b . . . HbA1c ≥8.0% (63.9 mmol/mol)
9. Previous allogeneic bone marrow transplant or solid organ transplant.

Key exclusion criteria for the phase III only:
1. Any prior treatment with AKT, PI3K or mTOR inhibitors.
2. Prior treatment with CDK4/6 inhibitors in the metastatic setting (prior CDK4/6 inhibitors permitted in the adjuvant setting provided there was a CDK4/6i treatment free interval of at least 12 months).
3. More than 1 line of chemotherapy for metastatic disease.

### Biomarker Rules

The PIK3CA/AKT1/PTEN-altered subgroup comprises all participants with a tumour carrying at least one genetic alteration in PIK3CA, AKT1, or PTEN as detected by FoundationOne®CDx (F1CDx) NGS Clinical Trial Assay (from Foundation Medicine, Cambridge, MA, USA) or Burning Rock OncoScreen Plus and classified according to the biomarker rules in Table A.

TABLE A
PIK3CA/AKT1/PTEN-altered Biomarker Rules

| Gene (transcript) | Variant class | Biomarker rules |
|---|---|---|
| PIK3CA (NM_006218) | Short variant | R88Q, N345K, C420R, E542K, |
| | | E545A, E545D, E545G, E545K, |
| | | E545Q, Q546E, Q546K Q546P, |
| | | Q546R, M1043V, M1043I, H1047L, |
| | | H1047R, H1047Y, and G1049R |
| | | alterations |
| AKT1 (NM_001014431) | Short variant | Any short variant with protein |
| | | effect E17K |
| PTEN (NM_000314) | Short variant | Any short variants listed below: |
| | | C124R, C124S, G129E, G129V, |
| | | G129R, R130Q, R130G, R130L, |
| | | R130P, C136R, C136Y, S170R, and |
| | | R173C |
| | | Any nonsense (including stop |
| | | codons), frameshift, or splice site |
| | | alteration, including those that |
| | | affect the start codon (ie, M1?, |
| | | M1T, M1fs*23) |
| | Copy number alteration | Any homozygous deletion of one |
| | | or more exons, regardless of |
| | | transcript |
| | Rearrangement | Any rearrangement that |
| | (Optional)^{a} | disrupts protein function, |
| | | regardless of transcript |
| | | Intragenic events including |
| | | duplications of only part of |
| | | the gene, deletions, or |
| | | inversions |
| | | Translocations, deletions, or |
| | | inversions where one |
| | | breakpoint is in PTEN and |
| | | the other breakpoint is in |
| | | another gene or intergenic |
| | | region |

^{a}PTEN rearrangement not included in OncoScreen Plus ™ biomarker rules.

### Results

### Study Population

At the time of data cut-off of 31 Oct. 2022, 39 patients had been treated with capivasertib in combination with palbociclib and fulvestrant across three dose levels. Dose 1 opened first; dose levels 2 and 3 opened simultaneously:
Dose level 1: capivasertib 320 mg+palbociclib 125 mg+fulvestrant 500 mg
Dose level 2: capivasertib 400 mg+palbociclib 100 mg+fulvestrant 500 mg
Dose level 3: capivasertib 400 mg+palbociclib 125 mg+fulvestrant 500 mg

Baseline characteristics of the 39 patients are shown in Table 8; characteristics were generally balanced across the dose levels.

Patients were heavily pre-treated (Table 9)
Approximately 80% of patients (n=31/39) had prior CDK4/6i exposure in the advanced setting, and 41% of patients (n=16/39) had received chemotherapy for advanced disease

The median duration of exposure for each agent is shown in Table 10
Greater exposure observed at dose level 1 reflects the longer follow-up relative to the other dose levels
Study treatment was ongoing in approximately half of the patients at the time of data cut-off

TABLE 8
Baseline Characteristics

| | | Capivasertib | | Capivasertib | | Capivasertib | | | |
|---|---|---|---|---|---|---|---|---|---|
| | | 320 mg + | | 400 mg + | | 400 mg + | | | |
| | | palbociclib | | palbociclib | | palbociclib | | | |
| | | 125 mg + | | 100 mg + | | 125 mg + | | | |
| | | fulvestrant 500 | | fulvestrant 500 | | fulvestrant 500 | | Total | |
| | | mg (n = 14) | | mg (n = 13) | | mg (n = 12) | | (N = 39) | |
| Female, n (%) | | 14 | (100) | 13 | (100) | 12 | (100) | 39 | (100) |
| Estrogen receptor-positive^{a} | | 13 | (92.9)^{a} | 11 | (84.6)^{b} | 12 | (100) | 36 | (92.3)^{a, b} |
| Progesterone receptor-positive | | 8 | (57.1) | 3 | (23.1) | 7 | (58.3) | 18 | (46.2) |
| Median age, years (range) | | 59.5 | (50-76) | 57.0 | (44-69) | 58.0 | (38-82) | 59.0 | (38-82) |
| Median BMI, kg/m² (range) | | 28.9 | (18-36) | 25.2 | (22-38) | 26.3 | (17-35) | 26.8 | (17-38) |
| Race, n (%)^{c} | White | 12 | (85.7) | 11 | (84.6) | 9 | (75.0) | 32 | (82.1) |
| | Missing | 2 | (14.3) | 2 | (15.3) | 3 | (25.0) | 7 | (17.9) |
| Ethnicity, | Hispanic or Latino | 1 | (7.1) | 0 | | 0 | | 1 | (2.6) |
| n (%) | Not Hispanic or Latino | 11 | (78.6) | 12 | (92.3) | 12 | (100) | 35 | (89.7) |

| | | | | | | | |
|---|---|---|---|---|---|---|---|
| Missing | 2 | (14.3) | 1 | (7.7) | 0 | 3 | (7.7) |

^{a}ER status was unknown for one patient;
^{b}ER status was negative for one patient and missing for one patient;
^{c}No patients were included in the Black or African American, Native Hawaiian or Other Pacific Islander, American Indian or Alaska Native, Asian, and Other race categories.

TABLE 9
Prior therapies

| Capivasertib | Capivasertib | Capivasertib | |
|---|---|---|---|
| 320 mg + | 400 mg + | 400 mg + | |
| palbociclib | palbociclib | palbociclib | |
| 125 mg + | 100 mg + | 125 mg + | |
| fulvestrant 500 | fulvestrant 500 | fulvestrant 500 | Total |
| mg (n = 14) | mg (n = 13) | mg (n = 12) | (N = 39) |

Prior therapies in the adjuvant and advanced setting, n (%)

| Immunotherapy | 0 | | 1 | (7.7) | 0 | | 1 | (2.6) |
|---|---|---|---|---|---|---|---|---|
| Hormonal therapy | 13 | (92.9) | 13 | (100) | 12 | (100) | 38 | (97.4) |
| Cytotoxic chemotherapy | 11 | (78.6) | 10 | (76.9) | 6 | (50.0) | 27 | (69.2) |
| Median prior chemotherapy | 2.5 | | 2.0 | | 1.0 | | 2.0 | |
| regimens | | | | | | | | |
| Targeted therapy | 9 | (64.3) | 9 | (69.2) | 5 | (41.7) | 23 | (59.0) |

Prior therapies in the advanced setting, n (%)

| CDK4/6i | 11 | (78.6) | 12 | (92.3) | 8 | (66.7) | 31 | (79.5) |
|---|---|---|---|---|---|---|---|---|
| Palbociclib | 6 | (42.9) | 8 | (61.5) | 7 | (58.3) | 21 | (53.8) |
| Ribociclib | 3 | (21.4) | 2 | (15.4) | 1 | (8.3) | 6 | (15.4) |
| Abemaciclib | 2 | (14.3) | 3 | (23.1) | 0 | | 5 | (12.8) |
| Fulvestrant | 7 | (50.0) | 7 | (53.9) | 3 | (25.0) | 17 | (43.6) |
| Chemotherapy | 6 | (42.9) | 7 | (53.9) | 3 | (25.0) | 16 | (41.0) |

TABLE 10
Median duration of exposure

| | | |
|---|---|---|
| Capivasertib | Capivasertib | Capivasertib |
| 320 mg + | 400 mg + | 400 mg + |
| palbociclib | palbociclib | palbociclib |
| 125 mg + | 100 mg + | 125 mg + |
| fulvestrant 500 | fulvestrant 500 | fulvestrant 500 |
| mg (n = 14) | mg (n = 13) | mg (n = 12) |

Total duration of exposure^{a}

| | | | |
|---|---|---|---|
| Capivasertib, median (range); months | 4.5 (0.3-15.8) | 3.1 (0.8-5.9) | 3.7 (1.7-8.9) |
| Palbociclib, median (range); months | 5.8 (1.6-15.9) | 3.0 (0.7-6.0) | 3.5 (1.7-9.0) |
| Fulvestrant, median (range); months | 6.0 (1.4-15.8) | 3.1 (0.9-5.7) | 3.7 (0.9-8.9) |

Patients ongoing study treatment

| | | | |
|---|---|---|---|
| Capivasertib, n (%) | 7 (50.0) | 6 (46.2) | 10 (83.3) |
| Palbociclib, n (%) | 8 (57.1) | 6 (46.2) | 10 (83.3) |
| Fulvestrant, n (%) | 7 (50.0) | 6 (46.2) | 10 (83.3) |

^{a}Data are immature.

### Safety

The most frequent AEs (occurring in ≥20% of patients in the total treatment group at any grade) are listed in Table 11. Most AEs were grade 1.

The type and incidence of AEs were as expected, given the known safety profile of the individual treatments, with no marked differences between the safety profile of the three dose levels.

There were no treatment-related deaths; one case of viral pneumonia with outcome death was considered unrelated to investigational medicinal products.

TABLE 11
Most frequent AEs by preferred term (occurring in ≥20%
patients in total treatment group at any grade)

| | Capivasertib 320 | | Capivasertib 400 | | Capivasertib 400 | | | |
|---|---|---|---|---|---|---|---|---|
| | mg + palbociclib | | mg + palbociclib | | mg + palbociclib | | | |
| | 125 mg + | | 100 mg + | | 125 mg + | | | |
| | fulvestrant 500 | | fulvestrant 500 | | fulvestrant 500 | | | |
| | mg (n = 14) | | mg (n = 13) | | mg (n = 12) | | Total (N = 39) | |
| | Any | | Any | | Any | | Any | |
| n, (%) | grade | Grade ≥3 | grade | Grade ≥3 | grade | Grade ≥3 | grade | Grade ≥3 |
| Neutropenia | 10 | 8 | 10 | 10 | 8 | 6 | 28 | 24 |
| | (71.4) | (57.1) | (76.9) | (76.9) | (66.7) | (50.0) | (71.8) | (61.5) |
| Diarrhoea | 9 | 0 | 9 | 1 | 9 | 0 | 27 | 1 |
| | (64.3) | | (69.2) | (7.7) | (75.0) | | (69.2) | (2.6) |
| Fatigue | 5 | 0 | 5 | 0 | 6 | 0 | 16 | 0 |
| | (35.7) | | (38.5) | | (50.0) | | (41.0) | |
| Nausea | 7 | 0 | 6 | 0 | 3 | 0 | 16 | 0 |
| | (50.0) | | (46.2) | | (25.0) | | (41.0) | |
| Vomiting | 5 | 0 | 3 | 0 | 4 | 0 | 12 | 0 |
| | (35.7) | | (23.1) | | (33.3) | | (30.8) | |
| Rash | 5 | 0 | 3 | 0 | 3 | 0 | 11 | 0 |
| | (35.7) | | (23.1) | | (25.0) | | (28.2) | |
| Urinary tract | 6 | 1 | 1 | 0 | 4 | 0 | 11 | 1 |
| infection | (42.9) | (7.7) | (7.7) | | (33.3) | | (28.2) | (2.6) |
| Blood | 4 | 1 | 1 | 0 | 4 | 0 | 9 | 1 |
| creatinine | (28.6) | (7.1) | (7.7) | | (33.3) | | (23.1) | (2.6) |
| increased | | | | | | | | |
| Decreased | 4 | 0 | 4 | 0 | 1 | 0 | 9 | 0 |
| appetite | (28.6) | | (30.8) | | (8.3) | | (23.1) | |
| Back pain | 5 | 0 | 2 | 0 | 1 | 0 | 8 | 0 |
| | (35.7) | | (15.4) | | (8.3) | | (20.5) | |
| Pruritus | 2 | 0 | 3 | 0 | 3 | 0 | 8 | 0 |
| | (14.3) | | (23.1) | | (25.0) | | (20.5) | |

### Dose-Limiting Toxicities (DLTs) and Recommended Phase 3 Dose (RP3D)

DLTs were seen in 6 of 35 evaluable patients, as shown in Table 12
Neutropenia/neutrophil count decreased (grade 3: n=5; grade 4: n=1) was the most frequent DLT and was driven by palbociclib
DLTs did not prevent dose escalation to the highest planned dose level of each agent and dose level expansion
Only one DLT (grade 3 neutropenia) was reported at the highest planned dose level of the triplet combination (capivasertib 400 mg+palbociclib 125 mg+fulvestrant 500 mg)

All three dose levels of the triplet combination were considered safe and tolerable; the highest planned dose level (capivasertib 400 mg+palbociclib 125 mg+fulvestrant 500 mg) was selected as the RP3D.

TABLE 12
DLTs.

| | Capivasertib 320 mg + | Capivasertib 400 mg + | Capivasertib 400 mg + |
|---|---|---|---|
| | palbociclib 125 mg + | palbociclib 100 mg + | palbociclib 125 mg + |
| | fulvestrant 500 mg | fulvestrant 500 mg | fulvestrant 500 mg |
| Dose level | (n = 14) | (n = 13) | (n = 12) |
| Evaluable patients^{a} | 3/12 | 2/12 | 1/11 |
| with DLT^{b}, n/N | | | |
| DLTs by patient | Neutrophil count | Neutropenia^{c} | Neutropenia^{c} |
| | decreased (grade 3) | (grade 3) | (grade 3) |
| | Neutropenia^{c} | Neutropenia^{c} | |
| | (grade 4) | (grade 3) | |
| | Blood creatinine | | |
| | increased (grade 2) | | |
| | Febrile neutropenia^{c} | | |
| | (grade 3) | | |

^{a}DLT-evaluable participants met key eligibility criteria and either experienced a DLT during Cycle 1 or received ≥65% of the assigned dose of all three study medications and completed Cycle 1;
^{b}Protocol-defined treatment-emergent AE that occurred during Cycle 1 and prevented further administration of capivasertib or CDK4/6i at the assigned dose level;
^{c}DLT definitions: febrile neutropenia (absolute neutrophil count <1000/μL with fever >101° F. [38.3° C.]); grade 4 neutropenia (absolute neutrophil count <500/μL) for >7 days; prolonged grade 3 neutropenia for >14 days.

### Pharmacokinetics (PK)

PK of capivasertib was consistent with previous monotherapy data.

It is estimated that capivasertib increased the palbociclib area under curve (AUC) by approximately 20% based on the observed and expected accumulation from Cycle 0, Day 1 to Cycle 1, Day 11 and Day 14
Day 11/Day 1 ratio was 2.89 (90% CI 2.64-3.15), and Day 14/Day 1 ratio was 2.77 (90% CI 2.54-3.03)
The expected ratio was 2.4 (Yu Y, et al. J Clin Pharmacol 2017; 57 (2): 173-184).

No clinically relevant drug-drug interaction was observed

### Clinical Efficacy

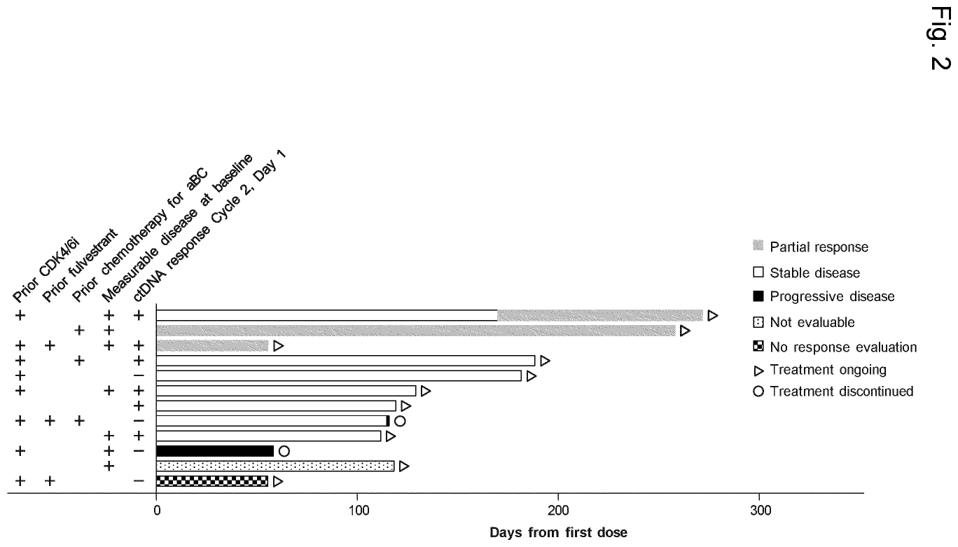

Preliminary response data indicate evidence of clinical activity in this heavily pre-treated population at the RP3D (FIG. 2)
Confirmed PRs to the triplet combination were observed, including in patients previously treated with CDK4/6i and/or fulvestrant

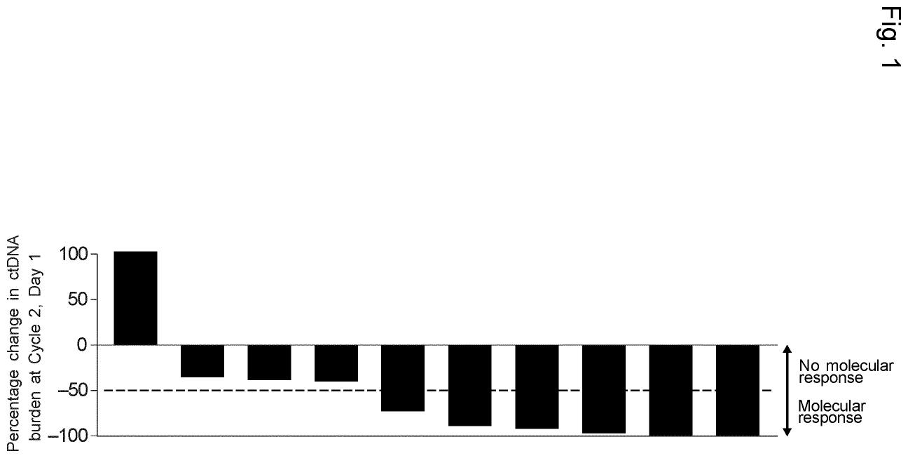

Evaluation of ctDNA in patients treated at the RP3D showed a >50% decrease in ctDNA burden at Cycle 2, Day 1 (vs screening) in 6 out of 10 evaluable patients, indicating a molecular response to the triplet combination (FIG. 1)

Accordingly, capivasertib 400 mg+palbociclib 125 mg+fulvestrant 500 mg was selected as the dose for the Phase III part of the study. In the Phase III part of the study, participants will be randomised in a 1:1 ratio to receive capivasertib or placebo (400 mg, oral twice daily, 4 days on and 3 days off for four weeks) plus palbociclib (125 mg, oral once daily, 3 of 4 weeks), and fulvestrant (500 mg IM monthly plus loading dose Cycle 1).

### Japanese Participants

The preliminary limited PK data from Japanese participants indicate that the mean palbociclib plasma exposure after 100 mg oral dosing in combination with capivasertib 400 mg is roughly similar to the mean exposure observed in participants from the rest of the world after 125 mg oral dosing in combination with capivasertib 400 mg; moreover, the incidence of DLTs (febrile neutropenia) led to palbociclib dose de-escalation from 125 mg to 100 mg.

Accordingly, for Japanese participants, the dose and schedule of capivasertib, palbociclib, and fulvestrant to be administered in the Phase III part is capivasertib 400 mg (BD 4 days on 3 days off/week of a 28-day cycle), palbociclib 100 mg; QD 21 days of a 28-day cycle), and fulvestrant 500 mg (IM monthly [Day 1 of a 28-day cycle] with a loading dose of 500 mg 2 weeks after the first dose).

### Capivasertib+Ribociclib+Fulvestrant

Compared with historical monotherapy data, preliminary modelling from clinical data indicates capivasertib AUC was 19% higher (95% CI; 1% to 43%) with ribociclib 400 mg, and 16% higher (95% CI; 0% to 34%) with ribociclib 600 mg. These findings are consistent with DDI modelling assuming capivasertib as a substrate of CYP3A4 and ribociclib acting as a moderate (at 400 mg) to strong (at 600 mg) CYP3A inhibitor. Ribociclib exposure was generally within model predictions from the published population PK model and consistent with DDI predictions based on capivasertib as a weak CYP3A inhibitor.

Clinically, a higher incidence of hyperglycemia (all CTCAE Grades and Grade 3 and higher) was observed and 1 DLT reported at ribociclib 400 mg and capivasertib 400 mg dose level. At the ribociclib 600 mg and capivasertib 320 mg dose level lower incidence of Grade 3 hyperglycemia was observed compared to the combination of ribociclib 600 mg and capivasertib 400 mg or ribociclib 400 mg and capivasertib 400 mg (16.6% vs 50% vs 47.6% respectively).

Accordingly, the dose and schedule of capivasertib, ribociclib, and fulvestrant to be administered in the Phase III part is capivasertib 320 mg (BD 4 days on 3 days off/week of a 28-day cycle), ribociclib 600 mg (QD 21 days of 28-day cycle), and fulvestrant 500 mg (IM monthly [Day 1 of a 28-day cycle] with a loading dose of 500 mg 2 weeks after the first dose).

In the Phase Ib part of the study, the triplet combination of capivasertib, ribociclib, and fulvestrant at the doses for the Phase III part of the study showed a consistent safety profile with toxicities as expected in relation to the individual drug components.

This description and its specific examples, while indicating certain embodiments, are intended for purposes of illustration only. This disclosure, therefore, is not limited to the illustrative embodiments described in this specification, and may be variously modified. In addition, it is to be appreciated that various embodiments that are, for clarity reasons, described in the context of separate embodiments, also may be combined to form a single embodiment. Conversely, various embodiments that are, for brevity reasons, described in the context of a single embodiment, also may be combined to form sub-combinations thereof.

Any publications referred to within the specification are hereby incorporated by reference.

1. A method of treating a patient having hormone receptor-positive (HR+), HER2-negative locally advanced or metastatic breast cancer, comprising administering to the patient a combination therapy comprising a first amount of capivasertib, a second amount of a CDK4/6 inhibitor and a third amount of fulvestrant, wherein the first amount, second amount and third amount together comprise a therapeutically effective amount.

2. The method of claim 1, wherein the first amount of capivasertib administered to the patient is 400 mg twice daily.

3. The method of claim 1 or 2, wherein the CDK4/6 inhibitor is selected from palbociclib, ribociclib and abemaciclib.

4. The method of claim 1 or 2, wherein the CDK4/6 inhibitor is palbociclib.

5. The method of claim 4, wherein the second amount of palbociclib administered to the patient is 125 mg once daily.

6. The method of claim 4, wherein the second amount of palbociclib administered to the patient is 100 mg once daily.

7. The method of claim 1, wherein the first amount of capivasertib administered to the patient is 320 mg twice daily.

8. The method of any one of claims 1, 2 and 7, wherein the CDK4/6 inhibitor is ribociclib.

9. The method of claim 8, wherein the second amount of ribociclib administered to the patient is 600 mg once daily.

10. The method of any one of claims 1 to 9, wherein the third amount of fulvestrant administered to the patient is 500 mg once monthly.

11. The method of claim 1, wherein the combination therapy is administered to the patient over a 28-day cycle in which 400 mg of capivasertib is orally administered to the patient twice daily on days 1 to 4, 8 to 11, 15 to 18, and 22 to 25; 125 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

12. The method of claim 1, wherein the combination therapy is administered to the patient over a 28-day cycle in which 400 mg of capivasertib is orally administered to the patient twice daily on days 1 to 4, 8 to 11, 15 to 18, and 22 to 25; 100 mg of palbociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

13. The method of claim 6 or claim 12, wherein the patient is a Japanese patient.

14. The method of claim 6 or claim 12, wherein the patient is an Asian patient.

15. The method of claim 1, wherein the combination therapy is administered to the patient over a 28-day cycle in which 320 mg of capivasertib is orally administered to the patient twice daily on days 1 to 4, 8 to 11, 15 to 18, and 22 to 25; 600 mg of ribociclib is orally administered to the patient once daily on days 1 to 21; and 500 mg of fulvestrant is intramuscularly administered to the patient once daily on days 1 and 15 of the first 28-day cycle, and once on day 1 of the 28-day cycle thereafter.

16. The method of any one of claims 1 to 15, wherein the patient has previously received endocrine therapy.

17. The method of any one of claims 1 to 16, wherein the patient's breast cancer has recurred or progressed during, or within twelve months of the patient completing, adjuvant endocrine therapy.

18. The method of any one of claims 1 to 16, wherein the patient's breast cancer has progressed during endocrine therapy for locally advanced or metastatic breast cancer.

19. The method of claim 16, 17 or 18, wherein the endocrine therapy is tamoxifen or an aromatase inhibitor.

20. The method of any one of claims 1 to 19, wherein the patient has previously received an oral SERD as an adjuvant therapy.

21. The method of any one of claims 1 to 20, wherein the patient has previously received therapy with fulvestrant.

22. The method of any one of claims 1 to 21, wherein the patient has previously received chemotherapy for advanced breast cancer.

23. The method of any one of claims 1 to 22, wherein the patient has previously received a CDK4/6 inhibitor as an adjuvant therapy.

24. The method of any one of claims 1 to 23, wherein the patient has not previously received a CDK4/6 inhibitor as a therapy for locally advanced or metastatic breast cancer.

25. The method of any one of claims 1 to 24, wherein the patient's tumour cells comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

26. The method of any one of claims 1 to 24, wherein the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

27. The method of any one of claims 1 to 26, wherein the patient exhibits a molecular response to the combination therapy after one 28-day cycle of treatment.

28. The method of any one of claims 1 to 26, wherein the ctDNA burden of the patient is reduced by more than 50% after one 28-day cycle of treatment with the combination therapy.

29. The method of any one of claims 1 to 28, wherein the combination therapy provides an ORR of 40% or more.

30. The method of any one of claims 1 to 29, wherein the combination therapy provides a CBR at 24 weeks of 40% or more.

31. A combination therapy comprising capivasertib, a CDK4/6 inhibitor and fulvestrant for use in the method of any one of claims 1 to 30.

32. Use of capivasertib in the manufacture of a medicament for use in the method of any one of claims 1 to 30.

33. A kit comprising (a) capivasertib, and (b) instructions for the use of capivasertib in the method of any one of claims 1 to 30.

34. A kit comprising (a) a combination therapy comprising capivasertib, a CDK4/6 inhibitor and fulvestrant, and (b) instructions for the use of the combination therapy in the method of any one of claims 1 to 30.
